(19) United States
(12) Patent Application Publication
(10) Pub. No.: US 2011/0029083 A1
(43) Pub. Date: Feb. 3, 2011

(54) Flexible Spinal Implant

(75) Inventors: Richard A. Hynes, Melbourne Beach, FL (US); Keith E. Miller, Germantown, TN (US); Virginia L. Richardson, Memphis, TN (US); Brian R. Thoren, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/533,877

(22) Filed: Jul. 31, 2009

(51) Int. Cl. A61F 2/44

(52) U.S. Cl. 623/17.16

(57) ABSTRACT

A flexible spinal implant for insertion into an intervertebral disc space for intervertebral stabilization is provided comprising a flexible implant section which enables bending of the implant body to facilitate insertion of the flexible spinal implant into the disc space via a spinal surgical procedure. The flexible spinal implant comprises a leading end, a trailing end and a flexible mid section connecting the leading end and the trailing end, wherein the implant is deformable at or about the flexible mid section to thereby permit a substantially straight entry of the implant into the disc space, and delivered to the selected disc space at a desired insertion angle of approach via a spinal surgical procedure. The implant can have a leading end comprising a curved or bullet shaped configuration, and the flexible mid section may be comprised of a flexible material.

### FIELD OF THE INVENTION

The present invention relates to medical devices such as spinal intervertebral implants implanted between adjacent vertebral bodies of a spinal column section, and more particularly to a flexible medical implant for intervertebral stabilization comprising a flexible implant section which enables bending or pliancy of the implant body to thereby facilitate insertion of the spinal implant at a selected disc space via a spinal surgical procedure.

### BACKGROUND

The spine is divided into four regions comprising the cervical, thoracic, lumbar, and sacrococcygeal regions. The cervical region includes the top seven vertebral bodies or members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The sacrum region includes five fused vertebral members S1-S5, with S1 being adjacent to L5. The vertebral members of the spine are aligned in a curved configuration that includes a cervical, thoracic and lumbosacral curve. Within the spine, intervertebral discs are positioned between the vertebral members and permit flexion, extension, lateral bending, and rotation. An intervertebral disc functions to stabilize and distribute forces between vertebral bodies. The intervertebral disc is comprised of the nucleus pulposus surrounded and confined by the annulus fibrosis.

Intervertebral discs and vertebral members are prone to injury and degeneration. Damage to the intervertebral discs and/or vertebral members can result from various physical or medical conditions or events, including trauma, degenerative conditions or diseases, tumors, infections, disc diseases, disc herniations, aging, scoliosis, other spinal curvature abnormalities or vertebra fractures. Damage to intervertebral discs can lead to pain, neurological deficit, and/or loss of motion. Damaged intervertebral discs may adversely impact the normal curvature of the spine, and/or lead to improper alignment and positioning of vertebrae which are adjacent to the damaged discs. Additionally, damaged discs may lead to loss of normal or proper vertebral spacing.

Various known surgical procedures, treatments and techniques have been developed to address medical problems associated with damaged or diseased intervertebral discs. One treatment is a fusion procedure which partially removes the center or nuclear area of a damaged disc and fuses adjacent vertebral members to prevent relative motion between the adjacent vertebral bodies. A section of the disc, annulus and nucleus, is removed or cut out to allow insertion of a spinal implant or spacer. The spacer may be used in conjunction with bone graft or allograft material which enables the adjacent vertebrae to grow and fuse together. Existing spinal implants assist in maintaining disc space height during the fusion process while at the same time, permitting or enabling an element of compression and selective movement of the implant within the disc space while vertebral fusion is taking place. The implant or spacer may also assist in imparting desired alignment or lordosis of the adjacent vertebral bodies.

As is known to persons of skill in the art, there are a variety of structures and configurations which can be used to obtain the desired vertebral body spacing and alignment such as spacers, implants or cages. These structures come in a variety of configurations, features, contours, geometries and sizes depending on the specific medical application or use. Further, implants can be inserted from a variety of insertion approaches, including for example anterior, posterior, anterolateral, lateral, direct lateral and translateral approaches.

In the area of surgical procedures for spinal implants at the L4-L5 or the L5-S1 level, an implant is often inserted in the disc space via either an anterior or posterior approach. Delivery and insertion of a spinal implant into the L4-L5 or L5-S1 disc space via a lateral approach can be done, but is less common and more difficult to perform than other procedures such as anterior or posterior procedures. One reason for the difficulty in inserting an implant at the L4-L5 or L5-S1 level via a later approach surgical procedure is the anatomical position of the iliac crest relative to the position of the L4-L5 or L5-S1 disc space level.

The anatomical position and curved nature of the iliac crest relative to the vertebral disc space at L4-L5 or L5-S1 makes the iliac crest a physical obstruction to direct or straight access to the L4-L5 or L5-S1 disc space in a lateral surgical approach procedure. The iliac crest's position prevents a direct or straight angle of approach for delivery, entry and insertion of a spinal implant into the L4-L5 or L5-S1 vertebral disc space. Additionally, at the L4-L5 or L5-S1 disc space levels, as well as higher lumbar spine levels, there is a complexity of neurological and vascular structures that cross the implant delivery approach path or implant path of insertion. In order to clear the obstructing iliac crest, and neurological and vascular structures, for implant insertion at L4-L5 or L5-S1, via a lateral or direct lateral approach, the implant is typically delivered to the disc space at some angled lateral angle of approach.

An additional difficulty in a lateral approach procedure is that since an implant is delivered at some lateral angle of approach, the implant being inserted arrives at L4-L5 or L5-S1 in an angled orientation. It would be easier and more convenient for the implant to enter the disc space in as nearly a direct or straight lateral approach as possible. In order to do this, an implant being inserted into the disc space will have to turn or navigate a corner at the entry of the L4-L5 or L5-S1 disc space so that the implant can enter the disc space in a substantially lateral approach orientation. A drawback of existing implants is that many are rigid or have inflexible physical configurations which prevent the implant from being able to be turned or navigated around a corner. The rigid aspect of existing implant configurations makes it difficult to use or impractical to insert these rigid implants via a lateral approach procedure at L4-L5 or L5-S1. Such difficulties limit the number of lateral approach implant procedures at L4-L5 or L5-S1 and the number of surgeons who can perform such a lateral approach implant procedure.

There is a need for an improved intervertebral implant, and method for inserting an implant between adjacent vertebral bodies using minimally invasive surgical techniques, that overcomes drawbacks and difficulties in delivering and inserting an implant at a desired or selected disc space via a spinal surgical procedure.

### SUMMARY

There is provided a flexible spinal implant for insertion into an intervertebral disc space for intervertebral stabilization comprising a flexible implant section which enables bending or pliancy of the implant body to thereby facilitate insertion of the spinal implant into the disc space via a via a spinal surgical procedure.

There is provided a flexible spinal implant for insertion into an intervertebral disc space comprising a flexible implant section which enables bending of the implant body to thereby facilitate insertion of the spinal implant via a spinal surgical procedure, including, among others, a direct lateral lumbar interbody fusion (DLIF) procedure, a posterior lumbar interbody fusion (PLIF) procedure or a transforaminal lumbar interbody fusion (TLIF) procedure.

There is also provided a spinal implant for insertion into a disc space comprising a leading end, a trailing end and a flexible mid section connecting the leading end and the trailing end, wherein the implant is deformable at the flexible mid section to thereby permit a substantially straight lateral entry of the implant into a selected disc space. The implant is delivered to the selected disc space at an insertion angle of approach. The implant can have a leading end comprising a bullet shaped configuration. Further, the flexible mid section may be comprised of flexible material, at least one pivoting connection or a spring mechanism.

There is further provided a spinal implant for insertion into a selected disc space comprising, a leading end, a trailing end, a flexible mid section connecting the leading end and the trailing end and a central implant aperture bounded by the leading end, the trailing end and the flexible mid section. In one aspect, the implant is delivered at a lateral insertion angle of approach via an implant insertion channel. The implant is deformable about the flexible mid section through interaction with the implant insertion channel to thereby permit substantially straight lateral entry of the implant into the selected disc space via a lateral approach. Further, the flexible mid section may be comprised of flexible material, at least one pivoting connection or a spring mechanism.

Disclosed aspects or embodiments are discussed and depicted in the attached drawings and the description provided below.

### BRIEF DESCRIPTION OF THE DRAWINGS

FIG. 1 illustrates an anterior view of a section of a vertebral column, the sacrum and ilium;

FIG. 2 illustrates an anterior partial view of FIG. 1 showing a partial insertion of a flexible spinal implant at disc space L5-S1 according to one embodiment of the present disclosure;

FIG. 3 illustrates a side view of a flexible spinal implant according to one embodiment of the present disclosure;

FIG. 4 illustrates a side view of the flexible spinal implant of FIG. 2;

FIG. 5A illustrates an isometric view of a flexible spinal implant according to another embodiment of the present disclosure;

FIG. 5B illustrates a side view of the flexible spinal implant of FIG. 5A in an implant insertion channel according to one embodiment of the present disclosure;

FIG. 5C illustrates an isometric view of a flexible spinal implant according to another embodiment of the present disclosure;

FIGS. 6A-6E illustrate side views of flexible implant mid sections according other embodiments of the present disclosure; and

FIG. 7 illustrates an isometric view of a flexible spinal implant according to another embodiment of the present disclosure.

### DETAILED DESCRIPTION

The present invention relates to medical devices such as spinal intervertebral implants implanted between adjacent vertebral bodies, and methods of use, and more particularly to a flexible spinal implant for intervertebral stabilization of a spinal disc space via insertion of the flexible implant at a desired disc space. For purposes of promoting an understanding of the principles of the invention, reference will now be made to one or more embodiments or aspects, examples, drawing illustrations, and specific language will be used to describe the same. It will nevertheless be understood that the various described embodiments or aspects are only exemplary in nature and no limitation of the scope of the invention is thereby intended. Any alterations and further modifications in the described embodiments or aspects, and any further applications of the principles of the invention as described herein are contemplated as would normally occur to one skilled in the art to which the invention relates.

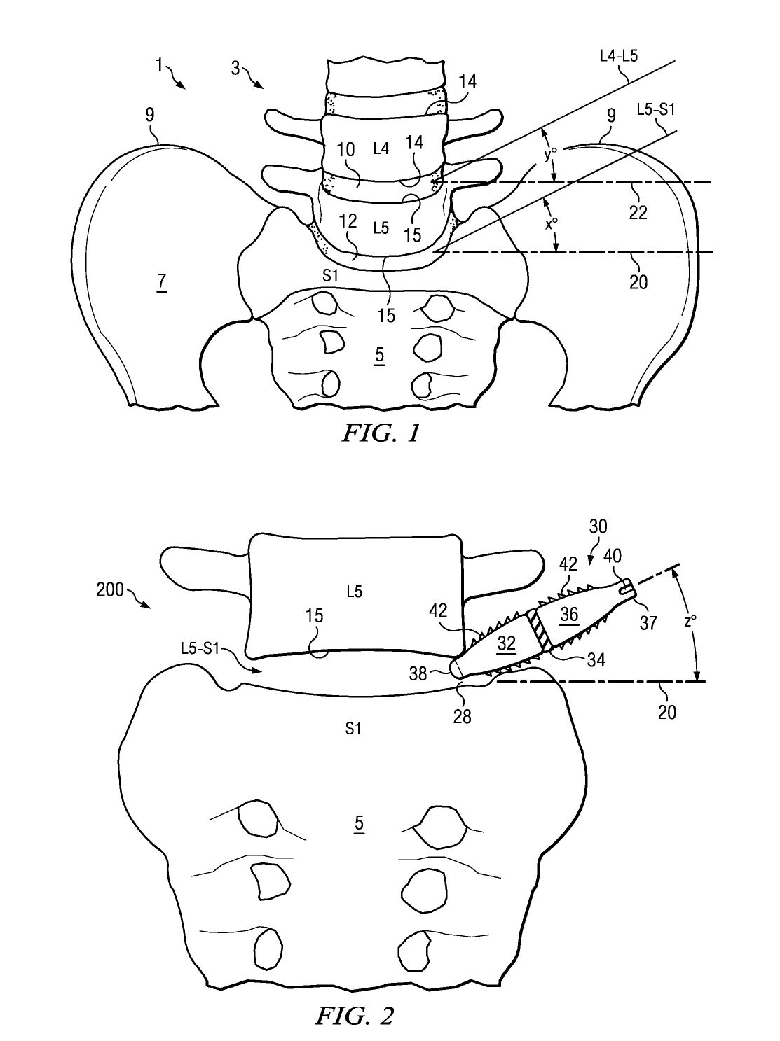

FIG. 1 shows an anterior view of a partial spinal section 1 of the vertebral column 3, the sacrum 5, ilium 7 and iliac crest 9. Also, shown are vertebral bodies L4, L5, Sacrum vertebrae 51, L4-L5 and L5-S1 vertebral disc spaces and the corresponding vertebral discs 10 and 12. The vertebral bodies L4 and L5 include end plates 14 and 15, respectively. FIG. 1 also shows straight or direct lateral reference lines 20 and 22 corresponding to the L5-S1 and L4-L5 disc spaces associated with a lateral approach procedure or lateral fusion surgical procedure. One lateral fusion surgical procedure for inserting an implant is known as a direct lateral interbody fusion (DLIF) procedure.

While FIGS. 1 and 2 illustrate a lateral approach surgical procedure, the flexible spinal implants contemplated and shown in FIGS. 1-7 may also be delivered and inserted into a desired disc space via other spinal surgical approaches and procedures as may be appropriate or required by a patient's anatomy or by a physician. For example, in one preferred aspect, the flexible spinal implant of the present disclosure can be delivered and inserted into a desired disc space via a lateral approach procedure such as a direct lateral lumbar interbody fusion (DLIF) procedure to clear the obstructing iliac crest, and neurological and vascular structures. And, in another preferred aspect, the flexible spinal implant may be delivered and inserted into a desired disc space via a posterior lumbar interbody fusion (PLIF) or transforaminal lumbar interbody fusion (TLIF) procedure to bend around and safely bypass or clear the cauda equina. In one preferred aspect, the flexible spinal implant is delivered via or through a minimal access spinal technology (MAST) surgical technique or procedure. Those of skill in the art will recognize that the flexible spinal implant may also be delivered and inserted via other known surgical approaches, including, a posterior, direct lateral, translateral, posterolateral, or anterolateral or any suitable oblique direction. Some known techniques and approaches that may be used to insert the flexible implant may also include, among others, anterior lumbar interbody fusion (ALIF). Further, those of skill in the art will recognize that a spinal implant may be delivered and inserted through other known surgical technique and procedures, including: open, mini-open or other minimally invasive surgical (MIS) techniques.

Referring to FIG. 1, in a lateral approach procedure, the physical position and configuration of the iliac crest 9 obstructs or prevents a direct or straight line 20 and 22 surgical approach to the L4-L5 or L5-S1 vertebral disc spaces for delivery and insertion of a spinal implant into the disc space. In order to overcome this drawback, and be able to laterally insert the implant at the L4-L5 or L5-S1 disc space via a lateral approach, the implant is delivered to the disc space at a lateral angle of approach, X or Y, relative to the straight lateral reference line 20 and 22. The lateral angle of approach X or Y for implant delivery is selected by a surgeon so as to clear or by-pass the obstructing iliac crest 9 encountered in a lateral approach procedure. Those of skill in the art will recognize that the lateral angle of approach Y corresponding to the L4-L5 disc space may be the same or different than the lateral angle of approach X corresponding to the L5-S1 disc space due to the different disc space positions relative to the iliac crest 9.

FIG. 2 shows an anterior partial view depicting a partial insertion of a flexible spinal implant 30 at disc space L5-S1 according to one aspect of the present disclosure. FIG. 4 also shows the flexible spinal implant 30 of FIG. 2. The flexible spinal implant 30 comprises a leading end 32, a trailing end 36 and a flexible mid section 34 connecting the leading end 32 and the trailing end 32. The flexible implant 30 also comprises anti-back out protrusions 42 on the upper and lower surfaces 50, 52, 54 and 56 of the flexible implant 30, and an instrument attachment section 40. Those of skill in the art will recognize that the anti-back out protrusions 42 extending from the upper and lower surfaces 50, 52, 54 and 56 will be configured and oriented so as to prevent the implant 30 from backing out or being ejected after implant insertion into the disc space. In the aspect shown in FIGS. 2 and 4, the anti-back out protrusions 42 have a triangular or pyramid configuration and are slanted or oriented back toward the trailing proximal implant end 37 of the implant 30.

The leading end 32 has a physical shape or physical configuration adapted to facilitate or ease implant insertion into the disc space L5-S1. In a preferred aspect, shown in FIG. 2, the leading end 32 has a curved or bullet shaped surface 38 which facilitates insertion of the flexible implant 30 in the L5-S1 disc space. The curved or bullet shaped nose 38 of the leading end 32 may, if the disc space is collapsed, impart a self-distracting force between the L5-S1 disc space which facilitates the insertion of flexible implant 30 into the L5-S1 disc space.

The trailing end 36 of the flexible implant 30 preferably comprises an implant grip or attachment section 40 situated at the proximal implant end 37 which enables the coupling of an insertion instrument (not shown). The attachment section 40 enables the controlled delivery of the flexible implant 30 into the L5-S1 disc space via a lateral surgical approach. In a preferred aspect, the attachment section 40 is recessed into the trailing end 36 such that when an instrument (not shown) is coupled to the flexible implant 30, the instrument is entirely interior to or flush with the exterior surface of the proximal implant end 37. In one aspect, the attachment section is a recessed slot 40 on both sides of the proximal implant end 37.

The flexible mid section 34 preferably connects the leading end 32 and the trailing end 36 to form the flexible spinal implant 30. The flexible mid section 34 is coupled or attached between the leading end 32 and the trailing end 36 so as to form a single assembled flexible spinal implant 30. The flexible mid section 34 is the aspect that permits or enables the implant to bend, flex or pivot at or about the flexible mid section 34 when the implant is being inserted into the L5-S1 disc space via a preferred later surgical approach.

The flexible mid section 34 also permits the implant 30 to be fully flexible in any one or more dimensional directions in space such that the flexible implant 30 can travel or rotate at or about the flexible section 34 to permit the flexible implant 30 to be delivered and inserted into the desired or selected disc space in a substantially straight approach orientation. In this manner, the flexible implant 30 is manufactured to have the physical properties or characteristics so that it can travel, bend or rotate about or at one or more reference lines, planes or axes A1, A2 and A3, e.g., as those shown and discussed with respect to FIG. 7. In this manner, the flexible implant 30, via the flexible mid section 34, can “self balance” or settle into or reach an equilibrium fit or best fit in the interbody disc space after implant insertion. The flexible mid section 34 permits the implant 30 to seek and find a fit or equilibrium fit after implant insertion through motion and/or micro motion and flexibility of the flexible section 34 until the flexible implant 30 finds the best anatomic fit in the disc space. This aspect of the flexible implant 30 enhances the biomechanical properties of the implant 30 while the vertebral fusion is setting. Those of skill in the art will recognize that in some embodiments, where required by a patient's anatomy or a physician's requirements, the flexible section 34, and by extension the flexible implant 30, can be manufactured so that the implant 30 is only flexible in a selected or desired linear dimensional direction or rotational direction in space during delivery and insertion into the desired or selected disc space in a substantially straight approach orientation.

Those of skill in the art will recognize that the flexible section 34 can be comprised of any bio compatible and flexible material that will permit the implant 30 to bend, deform, pivot or flex about or at the mid section 34. For example, it may be a deformable plastic, an elastic polymer, an elastomer, rubber or another deformable or elastic material. Further, in one aspect, the flexible implant 32 may be manufactured to have properties or characteristics such that such that the flexible mid section 34 can or will become rigid or substantially rigid once the implant is fully implanted in the disc space. The flexible implant section 34 can be manufactured to become rigid at a desired time or over time after implant insertion. For example, as soon as the implant is inserted in the disc space, over a desired or predetermined time period, or as the fusion is setting. The flexible implant 32 once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

In a lateral approach procedure, the flexible implant 30 arrives at the L5-S1 disc space entry 28 at the angle of approach or insertion angle of approach Z. Prior to implant insertion, the intervertebral disc space is typically prepared with a partial or complete discectomy in order to accept the flexible spinal implant which is to be inserted. In order to minimize damage to the vertebral bodies L5 and S1 and to facilitate entry of the implant into the L5-S1 disc space, it is preferred that the implant enter the L5-S1 disc space in a straight or substantially straight lateral approach orientation. Since the implant 30 arrives at the disc space entry 28 at the angle of approach Z, the implant must bend, deform or deflect such that the implant can enter the L5-S1 disc space in a substantially straight lateral approach orientation. The novel flexible mid section 34 enables or permits the flexible implant 30 to bend, deform or deflect at or about the flexible mid section 34 as needed to thereby enable or permit the substantially straight lateral approach entry of the implant 30 into the selected disc space when using a lateral approach procedure.

In this manner, the flexible implant 30 is adapted to bend and turn away from its delivery path orientation, having an insertion angle of approach Z, and enter the disc spaced L5-S1 in a substantially straight lateral approach orientation. As the flexible implant 30 is being delivered, via an instrument attached to the rear attachment section 40 (not shown), the leading end 32 of implant reaches and encounters an obstructing and opposing force at the S1 vertebrae at the disc space entry 28. That opposing force will tend to prevent or retard the entry of the implant into the disc space. This difficulty is overcome in a two fold manner. First, the curved or bullet shaped configuration 38 of the leading end 32 facilitates a smoother entry into the disc space L5-S1 and provides a curved or rounded contour that will facilitate entry and impart distraction of the vertebral bodies L5 and S1 as the implant continues to travel into the disc space. Secondly, the opposing force encountered due to the insertion angle of approach Z is translated through the leading end 32 to the flexible mid section 34. The flexibility or pliancy of the mid section 34 permits or enables the flexible implant 30 to bend, deform or deflect as needed about or at the flexible mid section 34. In this manner, the leading end 32 and the trailing end 36 of the flexible implant 30 will swing or rotate towards a straight lateral orientation that thereby permit the flexible implant to enter the L5-S1 disc space in a substantially straight lateral manner as the implant continues to be inserted or pushed into the disc space L5-S1 by a surgeon. The flexible implant 30, via the flexible mid section 34, will “self balance” and settle into or reach an equilibrium fit or best fit in the interbody disc space after implant insertion through motion and/or micro motion of the flexible section 34 until the flexible implant 30 settles into the he best anatomic fit in the disc space. Once the flexible implant 30 is inserted, the coupled instrument (not shown) can be disconnected from the attachment section 40. In one aspect, the flexible implant 32 will become rigid or substantially rigid once the implant is fully implanted in the disc space at desired time after implant insertion. For example, as soon as the implant is inserted in the disc space, over a desired or predetermined time period, or as the fusion is setting. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

In the depicted lateral approach of FIG. 2, the flexible implant 30 approaches or is delivered at an angle of approach Z measured relative to the straight lateral reference line 20. Those of skill in the art will recognize that the angle of approach may be a desired or selected insertion angle of approach depending on the disc space where a flexible implant is to be delivered via a lateral surgical procedure. The insertion angle of approach may be different depending on which spinal disc space level the implant is to be delivered at, e.g., the L4-L5 or L5-S1 disc space. Further, those of skill in the art will recognize that the insertion angle of approach may vary to accommodate a patient's or physician's needs and requirements during surgery. In one aspect, the desired insertion angle of approach is in the range of between five to forty-five degrees (5°-45°), with a preferred range of between ten and thirty degrees (10°-30°).

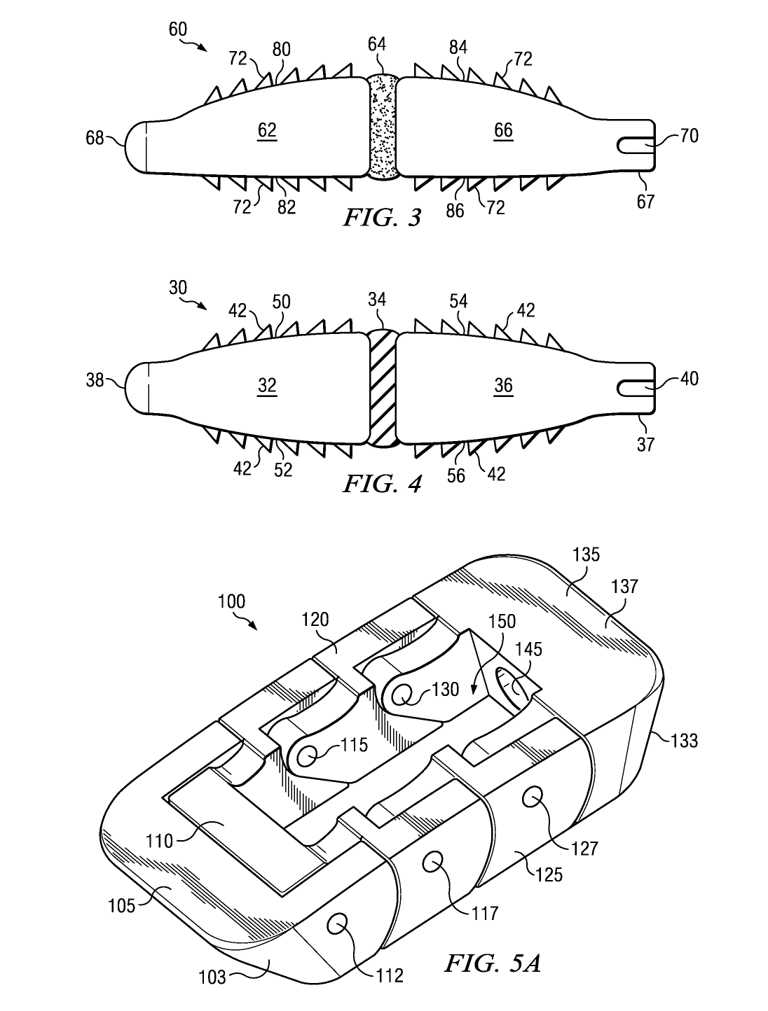

FIG. 3 shows a side view of a flexible spinal implant 60 according to another aspect of the present disclosure having a physical configuration adapted for implant 60 insertion at the L5-S1 disc space. The flexible spinal implant 60 comprises a leading end 62, a trailing end 66 and a flexible mid section 64 connecting the leading end 62 and the trailing end 32. The leading end 62 and the trailing end 66 respectively comprise curved or convex upper walls 80 and 84 to compliment the curved or concave nature of the L5 vertebral body end plate 15 when the implant is in place in the L5-S1 disc space. The leading end 62 and the trailing end 66 respectively comprise substantially flat lower walls 82 and 86 intended to compliment the relatively flat nature of the S1 sacral vertebrae when the implant is in place in the L5-S1 disc space. Those of skill in the art will recognize that other surface configurations, e.g., circular, oval, angled, etc., may be used instead depending on patient anatomy and physician requirements.

The flexible implant 60 further also comprises anti-back out protrusions 72 on the upper and lower surfaces 80, 82, 84 and 86, and an instrument attachment section 70. The anti-back out protrusions 72 extending from the upper and lower surfaces 80, 82, 84 and 86 will be configured and oriented so as to prevent the implant 60 from backing out or being ejected after insertion into the disc space. In the aspect shown in FIG. 3, the anti-back out protrusions 72 have a triangular configuration and are oriented back toward the trailing proximal implant end 67 of the implant 60.

The leading end 62 has a physical configurations adapted to facilitate insertion into the disc space L5-S1. In one aspect, the leading end 62 has a curved or bullet shaped surface 68 which facilitates insertion of the flexible implant 60 in the L5-S1 disc space. The curved or bullet shaped nose 68 will impart a distracting force between the L5-S1 disc space to facilitate insertion of the flexible implant 60. The trailing end 66 comprise an implant grip or attachment section 70 situated at the proximal implant end 77 which enables the coupling of an insertion instrument (not shown). The attachment section 70 enables for the controlled delivery of the flexible implant 70 into the L5-S1 disc space via a lateral approach. The attachment section 70 is preferably a recessed into the trailing end 66 such that when an instrument is coupled to the flexible implant 60, the instrument is entirely interior to the exterior surface of the proximal implant end 67. In one aspect, the attachment section is a recessed slot 70 on both sides of the proximal implant end 67.

The flexible mid section 64 preferably connects the leading end 62 and the trailing end 66 to form the flexible spinal implant 60. The flexible mid section 64 is coupled or attached between the leading end 62 and the trailing end 66 so as to form a single assembled flexible spinal implant 60. The flexible mid section 64 permits or enables the implant to bend, flex or pivot at or about the flexible mid section 64 when the implant is being inserted into the L5-S1 disc space via a preferred later surgical approach.

The flexible mid section 64 also permits the implant 60 to be fully flexible in any one or more dimensional directions in space such that the flexible implant 60 can travel or rotate at or about the flexible section 64 to permit the flexible implant 60 to be delivered and inserted into the desired or selected disc space in a substantially straight approach orientation. In this manner, the flexible implant 60 is manufactured to have the physical properties or characteristics so that it can travel, bend or rotate about or at one or more reference lines, planes or axes A1, A2 and A3, e.g., as those shown and discussed with respect to FIG. 7. In this manner, the flexible implant 60, via the flexible mid section 64, can “self balance” or settle into or reach an equilibrium fit or best fit in the interbody disc space after implant insertion. The flexible mid section 64 permits the implant 60 to seek and find a fit or equilibrium fit after implant insertion through motion and/or micro motion and flexibility of the flexible section 64 until the flexible implant 60 finds the best anatomic fit in the disc space. This aspect of the flexible implant 60 enhances the biomechanical properties of the implant 60 while the vertebral fusion is setting. Those of skill in the art will recognize that in some embodiments, where required by a patient's anatomy or a physician's requirements, the flexible section 64, and by extension the flexible implant 60, can be manufactured so that the implant 60 is only flexible in a selected or desired linear dimensional direction or rotational direction in space during delivery and insertion into the desired or selected disc space in a substantially straight approach orientation.

The flexible section 64 can be comprised of a biocompatible and flexible material that will permit the implant to bend or flex about or at the mid section 64. For example, a deformable plastic, an elastic polymer, an elastomer, rubber or another elastic material. In one aspect, the flexible implant 60 may be manufactured to have properties or characteristics such that such that the flexible mid section 64 can or will become rigid or substantially rigid once the implant is fully implanted in the disc space. The flexible implant section 64 can be manufactured to become rigid at a desired time or over time after implant insertion. For example, as soon as the implant is inserted in the disc space, over a desired or predetermined time period, or as the fusion is setting. In one aspect, the implant rigidity characteristic may be provided through the use of shape memory nitinol or other shape memory materials which can reach rigidity in a patient anatomical environment. The flexible implant 60 once rigid would thereafter no longer maintain implant flexibility. This aspect or property may be used where desired or required by a patient's anatomy or a physician's requirement.

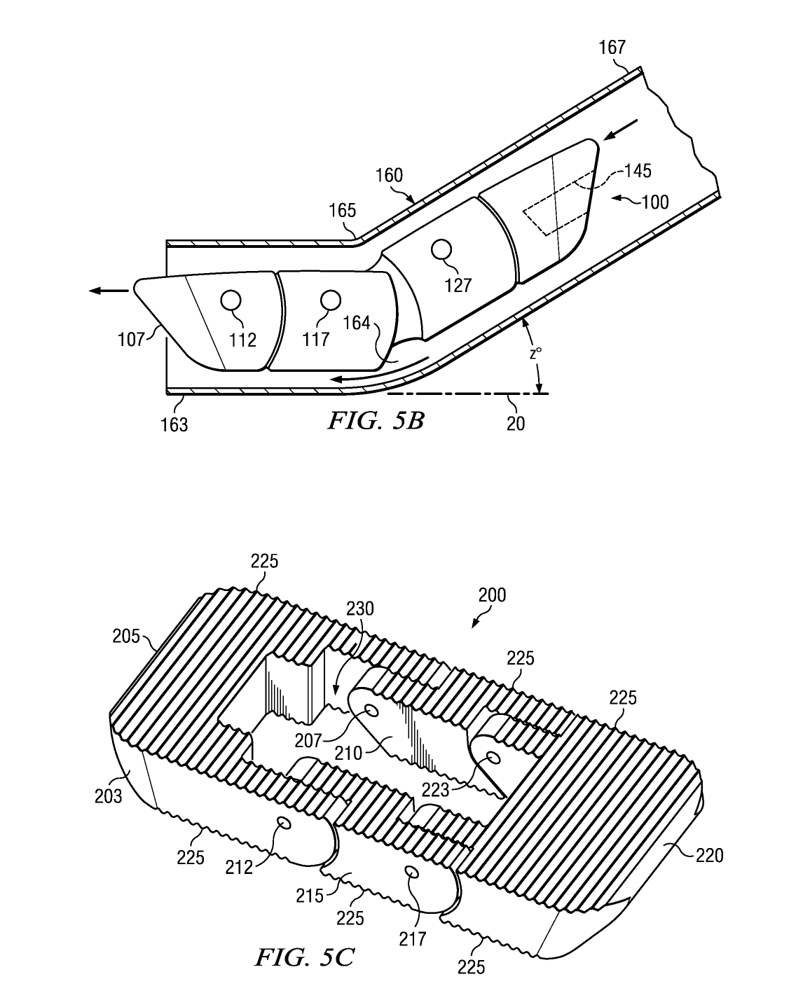

FIG. 5A shows an isometric view of a flexible spinal implant 100 according to another aspect of the present disclosure. FIG. 5B shows a side view of the flexible spinal implant 100 of FIG. 5A in an implant insertion channel 160 that can be positioned for implant insertion at a selected disc space, e.g., L4-L5 or L5-S1 shown in FIG. 1, via a lateral approach procedure. The flexible spinal implant 100 is a multi-component pivoting assembly comprising a leading end 105, a first member 110, a second member 120, a third member 125 and a trailing end 135.

The leading end 105 is pivotally connected to the first member 110 at a first hinge 112 to thereby permit rotational motion between the leading end 105 relative to the first member 110. The first member 110 is pivotally connected to the second member 120 at a second hinge 115 to thereby permit rotational motion of the first member 110 relative to the second member 120. The first member 110 is pivotally connected to the third member 125 at a third hinge 117 to thereby permit rotational motion of the first member 110 relative to the third member 125. The trailing end 135 is pivotally connected to the second member 120 at a fourth hinge 130 to thereby permit rotational motion of the trailing end 135 relative to the second member 120. The trailing end 135 is pivotally connected to the third member 125 at a fifth hinge 127 to thereby permit rotational motion of the trailing end 110 relative to the third member 125.

As shown in FIGS. 5A and 5B, the leading end 105 has a physical configuration adapted to facilitate or ease insertion of the flexible implant 100 into a disc space. In a preferred aspect, shown in FIGS. 5A and 5B, the leading end 105 has a wedge type shape 103 which facilitates insertion of the flexible implant 100 into a disc space. The wedge shaped nose 103 of the leading end 105 may, if the disc space is collapsed, impart a distracting force to adjacent collapsed vertebrae as the flexible implant 100 travels or is inserted into the disc space.

The trailing end 135 comprises an implant grip or attachment aperture 145 situated at the proximal implant end 137 which enables the coupling of an instrument (not shown) to the flexible spinal implant 100. The attachment aperture 145 enables an instrument to couple to the flexile spinal implant 100 for delivery of the flexible implant 100 through an implant insertion channel 160 into a selected disc space via a lateral approach. After insertion of the flexible implant 100, the attachment aperture 145 can also be used to insert graft material, as already discussed previously, if none was packed in prior to implant insertion.

The flexible spinal implant 100 further comprises an interior implant aperture 150 defined and formed by the pivotally connected first member 110, second member 120, third member 125 and trailing end 135. The interior implant aperture 150 can be filled or packed with graft material before or after insertion of the flexible implant 100 into the selected disc space. The graft material may be composed of material that has the ability to promote, enhance and/or accelerate bone growth and fusion of vertebral bodies. Graft material may include allograft material, bone graft, bone marrow, demineralized bone matrix putty or gel and/or any combination thereof. The filler graft material may promote bone growth through and around the interior implant aperture 150 to promote fusion of the disc space intervertebral joint. Those of skill in the art will recognize that the use of filler graft material is optional, and it may or may not be used depending on the needs or requirements of a physician or a medical procedure.

The first member 110, a second member 120 and a third member 125 are pivotally connected to each other and to the leading end 105 and trailing end 135 to form the multi-piece flexible implant 100 shown in FIGS. 5A and 5B. The pivoting connections 112, 115, 117, 127 and 130 permit or enable the flexible spinal implant 100 to pivot or articulate about the pivoting connections 112, 115, 117, 127 and 130 such that the flexible spinal implant 100 can bend and articulate as may be need to permit delivery and insertion of the flexible implant 100 in a lateral approach. For example, implant 100 insertion into disc space L4-L5 or L5-S1 shown in FIGS. 1 and 2. Those of skill in the art will recognize that the flexible implant 100 can have a different number of implant components and corresponding pivoting connections, e.g., as shown in FIG. 5C. The number of pivoting connections will depend on the angle of approach Z that the flexible implant 100 will be inserted at, or an insertion channel bend or turn 165 that the flexible implant 100 will traverse as the flexible implant 100 travels through the insertion channel 160. In one aspect, the greater the approach angle Z, the larger the number of implant components and corresponding pivoting connections required to enable the implant to sufficiently articulate in order to traverse the insertion channel bend 165. In one aspect, the flexible implant 100 may be manufactured to have properties or characteristics such that such that the pivoting connections 112, 115, 117, 127 and 130 can or will become rigid or substantially rigid once the implant 100 is fully implanted in the disc space. The pivoting connections 112, 115, 117, 127 and 130 can be manufactured to become rigid at a desired time or over time after implant insertion. For example, as soon as the implant is inserted in the disc space, over a desired or predetermined time period, or as the fusion is setting. The pivoting connections 112, 115, 117, 127 and 130 once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

The flexible implant 100 of FIG. 5A is preferably delivered to a disc space via an adjacently positioned implant insertion channel 160 in a lateral approach procedure. The following contemplates a delivery at the L4-L5 or L5-S1 disc space. However, those of skill in the art will recognize that the insertion described below may be carried out at other spinal disc levels. An implant insertion channel 160 is positioned adjacent the disc space L4-L5 or L5-S1 where the flexible implant 100 is to be inserted. The implant insertion channel 160 comprises a distal first channel end 163, a channel turn section 164 and a proximal second channel end 167. The implant insertion channel 160 is preferably a channel with a cross section that compliments the exterior physical configuration of the flexible implant 100 that will travel inside the insertion channel 160. In a preferred aspect, the implant insertion channel 160 has a rectangular cross-section. However, other configurations may be used, as appropriate, to compliment the flexible implant 100, e.g., circular, squared, etc.

The implant insertion channel 160 transitions from the first channel end 163 to the channel turn section 164 and then to the second channel end 167, as shown in FIG. 5B. The channel turn section 164 is oriented such that the first channel end 163 and the second channel end 167 describe an angle of approach Z. The channel turn section 164 is typically fixed. However, those of skill in the art will recognize that the channel turn section 164 could also be adjustable. For example, through a hinged arrangement between the first channel end 163 and the second channel end 167. In this manner, the implant insertion channel 160 could be adjusted to define a variety or range of angles of approach Z measured relative to the straight lateral reference line 20. The angle of approach Z may differ depending on the disc space where a flexible implant 100 is to be delivered and inserted via a lateral surgical procedure. The insertion angle of approach Z may also vary to accommodate a patient's or physician's needs and requirements during surgery. In one aspect, the desired insertion angle of approach Z is between five to forty-five degrees (5°-45°), with a preferred range of between ten and thirty degrees (10°-30°).

In a preferred aspect, the flexible implant 100 travels inside the implant insertion channel 160, as shown in FIG. 5B, to reach the desired or selected disc space. In order for the flexible implant 100 to reach and enter the disc space in a substantially straight lateral approach orientation, the traveling flexible implant 100 will be guided by the interior walls of the implant insertion channel 160. The channel turn section 164 interacts with and forces the flexible implant 100 to actuate and pivot about the pivoting connections 112, 115, 117, 127 and 130 as the flexible implant 100 travels through the channel turn section 164. This interaction imparts a force to the flexible spinal implant 100 such that the flexible spinal implant 100 articulates to thereby enable travel through the channel turn section 164. In this manner, the flexible implant 100 is adapted to articulate and turn from its delivery path having an insertion angle of approach Z and enter the disc space in a substantially straight lateral approach orientation. The forced articulation by the channel turn section 164 interaction, in particular, transitions the flexible implant 100 from an angled lateral approach Z at the second channel end 167 to a substantially straight lateral approach orientation in the first channel end 163, as shown in FIG. 5B. As the flexible implant 100 continues to travel inside the implant insertion channel 160, the flexible implant 100 will enter the selected disc space in a substantially lateral approach orientation. The flexible implant 100, via the pivoting connections 112, 115, 117, 127 and 130, will “self balance” and settle into or reach an equilibrium fit or best fit in the interbody disc space after implant insertion through motion and/or micro motion of the pivoting connections 112, 115, 117, 127 and 130 until the flexible implant 1000 comes to the best anatomic fit in the disc space. The flexible implant 100 can be delivered via an instrument (not shown) coupled to the attachment aperture 145 via travel through the implant insertion channel 160 into the selected disc space. Once the flexible implant 100 is inserted in the disc space, the coupled instrument can be disconnected from the attachment aperture 145.

FIG. 5C shows an isometric view of a flexible spinal implant 200 according to another embodiment of the present disclosure that can be inserted at a selected disc space, e.g., L4-L5 or L5-S1, via a lateral approach procedure. The flexible spinal implant 200 is a multi-component implant pivoting assembly comprising a leading end 205, a first member 210, a second member 215 and a trailing end 220. The leading end 205 is pivotally connected to the first member 210 at a first hinge 207 to thereby permit rotational motion between the leading end 205 relative to the first member 210. The leading end 205 is pivotally connected to the second member 215 at a second hinge 212 to thereby permit rotational motion between the leading end 205 relative to the second member 215. The trailing end 220 is pivotally connected to the first member 210 at a third hinge 223 to thereby permit rotational motion of the trailing end 220 relative to the first member 210. The trailing end 220 is pivotally connected to the second member 215 at a fourth hinge 217 to thereby permit rotational motion of the trailing end 220 relative to the second member 215.

The leading end 205 has a physical configuration adapted to facilitate or ease insertion of the flexible implant 200 into a disc space. In a preferred aspect, shown in FIG. 5C, the leading end 205 has a wedge type contour 203 which facilitates insertion of the flexible implant 200 into a disc space. The wedge shaped nose 203 of the leading end 205 may serve to impart a distracting force to adjacent vertebrae as the flexible implant 200 travels or is inserted into a disc space.

The flexible spinal implant 200 further comprises an interior implant aperture 230 defined by the pivotally connected leading end 205, first member 210, second member 215 and trailing end 220. The interior implant aperture 230 can be filled with a graft material before insertion of the flexible implant 200 into a selected disc space. The graft material may be composed of material that has the ability to promote, enhance and/or accelerate bone growth and fusion of vertebral bodies. The graft material may promote bone growth through and around the interior implant aperture 230 to promote fusion of the disc space intervertebral joint. The use of filler graft material is optional, and it may or may not be used depending on the needs or requirements of a physician or a medical procedure.

The flexible implant 200 also comprises anti-back out protrusions 225 on the upper and lower surfaces of the flexible implant 200. The anti-back out protrusions 225 extending from the upper and lower surfaces are preferably configured and oriented so as to prevent the implant 200 from backing out or being ejected after insertion into a disc space. In the aspect shown in FIG. 5C, the anti-back out protrusions 225 have a triangular ridge configuration that traverse across the upper and lower surfaces of the leading end 205, the first member 210, the second member 215 and the trailing end 220 of the flexible implant 200. Those of skill in the art will recognize that the protrusions can have other shapes, configurations or sizes including, among others, pyramids, triangles, cones, spikes and keels.

The first member 210 and second member 215 are pivotally connected to each other and to the leading end 205 and trailing end 220 to form the multi-component flexible implant 200 shown in FIG. 5C. The pivoting connections 207, 212, 217 and 223 permit or enable the flexible spinal implant 200 to pivot or articulate about the pivoting connections 207, 212, 217 and 223 such that the flexible spinal implant 200 can bend and articulate as may be needed to permit delivery and insertion of the flexible implant 200 in a disc space via a lateral approach. For example, into disc space L4-L5 or L5-S1 shown in FIGS. 1 and 2. As discussed previously, the flexible implant 200 can have a different number of implant components and corresponding pivoting connections. The number of pivoting connections will depend on the angle of approach Z that the flexible implant 200 will be inserted at. In one aspect, the flexible implant 200 may be manufactured to have properties or characteristics such that such that the pivoting connections 207, 212, 217 and 223 can or will become rigid or substantially rigid once the implant 200 is fully implanted in the disc space. The pivoting connections 207, 212, 217 and 223 can be manufactured to become rigid at a desired time or over time after implant insertion. For example, as soon as the implant is inserted in the disc space, over a desired or predetermined time period, or as the fusion is setting. The pivoting connections 207, 212, 217 and 223 once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

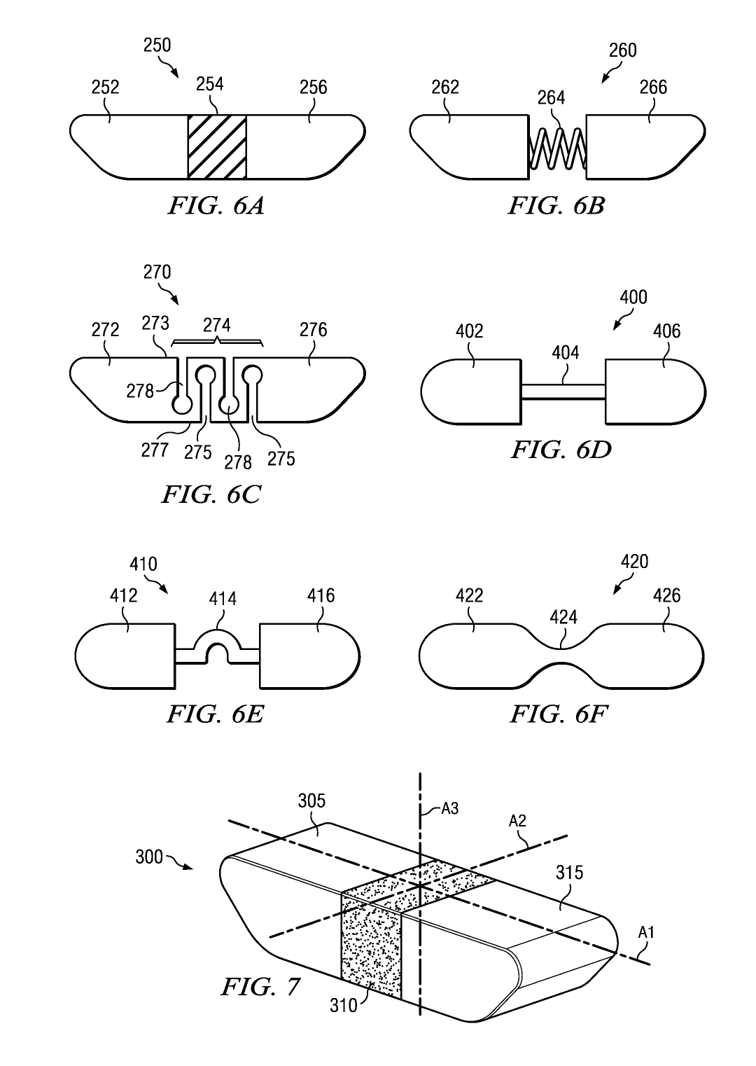

FIGS. 6A-6E show side views of flexible spinal implants 250, 260, 270, 400 and 410 which disclose other flexible mid section aspects 254, 264, 274, 404 and 410 contemplated in the present disclosure. The flexible mid sections 254, 264, 274, 404 and 410 enable the respective flexible implants 250, 260, 270, 400 and 410 to bend, flex or pivot at or about the flexible mid section 254, 264, 274, 404 and 410 so that an implant can enter the disc space in a substantially lateral approach orientation. The flexible implant 250, 260, 270, 400 and 410, via the flexible mid section 254, 264, 274, 404 and 410, will “self balance” and settle into or reach an equilibrium fit or best in the interbody disc space after implant insertion through motion and/or micro motion of the flexible section 254, 264, 274, 404 and 410 until the flexible implant 250, 260, 270, 400 and 410 comes to the best anatomic fit in the disc space. The flexible implants 250, 260, 270, 400 and 410, shown in FIGS. 6A-6E, are preferably delivered to a disc space, such as L4-L5 or L5-S1, in a lateral approach procedure. However, those of skill in the art will recognize that the insertion described below may be applied at other spinal disc level.

FIG. 6A shows a flexible spinal implant 250 comprising a leading end 252, a trailing end 256 and a flexible mid section 254 connecting the leading end 252 and the trailing end 256. The flexible section 254 can be comprised of any bio compatible and flexible material that permits the implant to bend or flex about or at the mid section 254, including a deformable plastic, an elastic polymer, an elastomer, rubber or another elastic material. FIG. 6B shows a flexible spinal implant 260 comprising a leading end 262, a trailing end 266 and a flexible mid section 264 connecting the leading end 262 and the trailing end 266. The flexible section 264 shown in FIG. 6B is contemplated as a flexible metallic section that is bio compatible and made of resilient flexible metallic material that permits the implant to bend or flex about or at the flexible mid section 264. In the aspect, shown in FIG. 6B, the flexible section 264 is a spring type mechanism that is bio compatible and which permits the implant 260 to bend or flex about or at the mid section 264.

FIG. 6C shows a flexible spinal implant 270 comprising a leading end 272, a trailing end 276 and a flexible mid section 274 connecting the leading end 272 and the trailing end 276. The flexible section 274 is contemplated as a series of slots 275 and 278 formed in the implant body to form the flexible section 274. The implant slots 275 and 278 form the flexible section that permit the implant 270 to bend or flex about or at the mid section 274. In another aspect, the mid section 274 may be comprised of a different number of slots 275 and 278. In another aspect, the mid section 274 may be comprised of slots 275 that are formed in the lower implant surface 277 or slots 278 that are formed in the upper implant surface 273.

FIGS. 6D and 6E show a flexible spinal implant 400 and 410 comprising a leading end 402 and 412, a trailing end 406 and 416 and a flexible mid section 404 and 414 connecting the leading end 402 and 412 and the trailing end 406 and 416. The flexible section 404 and 414 shown in the aspects of FIGS. 6D and 6E are a flexible metallic section that is bio compatible and made of resilient flexible metallic material that permits the implant 400 and 410 to bend or flex about or at the flexible mid section 404 and 414. In the aspect shown in FIG. 6D, the flexible section 404 is a flat metallic section or a leaf spring mechanism that is bio compatible and which permits the implant 400 to bend or flex about or at the mid section 404. In the aspect shown in FIG. 6E, the flexible section 4014 is a curved metallic section that is bio compatible and which permits the implant 410 to bend or flex about or at the mid section 414. The flexible metallic section 404 and 414 can be comprised of a biocompatible metallic material such as, among others, stainless steel, titanium, nitinol, platinum, tungsten, silver, palladium, cobalt chrome alloys, shape memory nitinol and mixtures thereof. The biocompatible metallic material used can depend on the patient's need and physician requirements.

FIG. 6F shows a flexible spinal implant 420 comprising a leading end 422, a trailing end 426 and a flexible mid section 424 connecting the leading end 422 and the trailing end 426. The flexible section 424 is contemplated as flexible section 424 which formed as part of the implant body 420 as a reduced size mid section or thin implant section 424 in the implant body 420 to form the flexible section 274. The reduced size mid section or thin implant section 424 forms the flexible section 424 which is manufactured to permit the implant 420 to bend or flex about or at the mid section 424.

In a further aspect contemplated for the flexible implants 250, 260, 270, 400, 410 and 420, shown in FIGS. 6A-6F, the flexible implant 250, 260, 270, 400, 410 and 420 may be manufactured to have properties or characteristics such that such that the flexible mid section 254, 264, 270, 404, 414 and 424 can or will become rigid or substantially rigid once the implant is fully implanted in the disc space. The flexible implant section 254, 264, 270, 404, 414 and 424 can be manufactured to become rigid at a desired time or over time after implant insertion. For example, as soon as the implant is inserted in the disc space, over a desired or predetermined time period, or as the fusion is setting. The flexible implant 250, 260, 270, 400, 410 and 420 once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

FIG. 7 shows an isometric view of a flexible spinal implant 300 according to a further aspect the present disclosure. Similar to flexible implant aspects already discussed, the flexible mid section 310 enables or permits the flexible implant 300 to bend, flex or pivot at or about the flexible mid section 310 so that an implant can enter the disc space in a substantially straight approach orientation, e.g., a straight lateral approach orientation. The flexible implant 300 may be delivered to a selected disc space such as L4-L5 or L5-S1 discussed herein or other desired spinal disc levels. The flexible spinal implant 300 can be delivered and inserted into a desired disc space via a spinal surgical approach and procedure selected or required by a physician.

The flexible spinal implant 300 can be delivered and inserted into a desired disc space via a lateral approach procedure such as a DLIF procedure to clear the obstructing iliac crest, and neurological and vascular structures. The flexible spinal implant 300 may also be delivered and inserted into a desired disc space via a PLIF or TLIF procedure to bend around and safely bypass or clear the cauda equina. In one aspect, the flexible spinal implant 300 is delivered via or through a minimal access spinal technology (MAST) surgical technique or procedure. Those of skill in the art will recognize that the flexible spinal implant 300 may also be delivered and inserted via other known surgical approaches, including, a posterior, direct lateral, translateral, posterolateral, or anterolateral or any suitable oblique direction. Some known techniques and approaches that may be used to insert the flexible implant 300 may also include, among others, anterior lumbar interbody fusion (ALIF). Further, those of skill in the art will recognize that a spinal implant may be delivered and inserted through known surgical technique and procedures, including: open, mini-open or other minimally invasive surgical (MIS) techniques.

FIG. 7 shows a flexible spinal implant 300 comprising a leading end 305, a trailing end 315 and a flexible mid section 310 connecting the leading end 305 and the trailing end 315. FIG. 7 additionally shows three dimensional (3D) implant reference lines, planes or axes A1, A2 and A3. The 3D reference implant reference lines, planes or axes A1, A2 and A3 may be a selected or desired reference line, plane or axis. Those of skill in the art will recognize that the 3D reference implant reference lines, planes or axes A1, A2 and A3 may also or instead be known references lines, planes or axes such as the traditional x-y-z axes, or line, planes or axes that represent Axial, Sagittal or Coronal anatomical planes. The flexible section 310 can be comprised of any bio compatible and flexible material that permits the implant to bend or flex about or at the mid section 310, including a deformable plastic, an elastic polymer, an elastomer, rubber or another elastic material.

The flexible section 310, shown in FIG. 7, further illustrates another advantageous aspect of the flexible implant 300. The flexible section 310 permits the implant to travel, bend or flex about or at the mid section 310, in or along any one of the 3D reference implant reference lines, planes or axes A1, A2 and A3 as the implant 300 is being delivered and inserted into the desired or selected disc space, or as the implant 300 is swinging, bending or turning away from its delivery path orientation to thereby enter the disc space in a substantially straight approach orientation. The flexible section 310 also permits the implant 300 to travel, bend or flex about or at the mid section 310, in any three dimensional direction or orientation with respect to the 3D reference implant reference lines, planes or axes A1, A2 and A3 as the implant 300 is being delivered and inserted into the desired or selected disc space, or as the implant is swinging, bending or turning away from its delivery path orientation to thereby enter the disc space in a substantially straight approach orientation. The flexible section 310 further permits the implant 300 to rotate, travel, bend or flex about or at the mid section 310, in any one or more dimensional direction or orientation with respect to the 3D reference implant reference lines, planes or axes A1, A2 and A3 as the implant 300 is being delivered and inserted into the desired or selected disc space, or as the implant 300 is swinging, bending or turning away from its delivery path orientation to thereby enter the disc space in a substantially straight approach orientation.

The flexible section 310 thereby permits the implant 300 to be fully flexible, deformable or moveable in any one or more dimensional directions in space such that the flexible implant 300 can travel or rotate at or about the flexible section 310 to permit the flexible implant 300 to be delivered and inserted into the desired or selected disc space in a substantially straight approach orientation. In this manner, the flexible implant 300 has the physical properties or characteristics so that it can travel or rotate about or at one or more reference lines, planes or axes A1, A2 and A3. In this manner, the flexible implant 300, via the flexible section 310 can “self balance” or settle into or reach an equilibrium fit or best fit in the interbody disc space after implant 300 insertion. The flexible implant section 310 permits the implant 300 to reach a fit or equilibrium fit after implant insertion through motion and/or micro motion and flexibility of the flexible section 310 until the flexible implant 300 settles into the best anatomic fit in the disc space. This aspect of the flexible implant 300 enhances the biomechanical properties of the implant 300 while the vertebral fusion is setting. This novel aspect discussed with respect to FIG. 7 is also contemplated for the flexible implants previously discussed in relation to FIGS. 1-6C. Those of skill in the art will recognize that in some embodiments, where required by a patient's anatomy or a physician's requirements, the flexible section 310, and by extension the flexible implant 300, could be manufactured so that the implant 300 is only flexible in a selected or desired linear dimensional direction or rotational direction in space during delivery and insertion into the desired or selected disc space in a substantially straight approach orientation.

In a further aspect contemplated for the flexible implants depicted and discussed with respect to FIGS. 1-7, the flexible implant may be manufactured to have properties or characteristics such that such that the flexible mid section can or will become rigid or substantially rigid once the implant is fully implanted in the disc space. The flexible implant section can be manufactured to become rigid at a desired time or over time after implant insertion. For example, as soon as the implant is inserted in the disc space, over a desired or predetermined time period, or as the fusion is setting. The flexible implant once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

The flexible implants disclosed in this disclosure are preferably comprised of biocompatible materials substrates which can be attached to the novel flexible implant sections to form a whole flexible spinal implant. The biocompatible material substrate may include, among others, polyetheretherketone (PEEK) polymer material, homopolymers, co-polymers and oligomers of polyhydroxy acids, polyesters, polyorthoesters, polyanhydrides, polydioxanone, polydioxanediones, polyesteramides, polyaminoacids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof. The biocompatible material substrate may also be a metallic material and may include, among others, stainless steel, titanium, nitinol, platinum, tungsten, silver, palladium, cobalt chrome alloys, shape memory nitinol and mixtures thereof. The biocompatible material used can depend on the patient's need and physician requirements.

While embodiments of the invention have been illustrated and described in detail in the present disclosure, the disclosure is to be considered as illustrative and not restrictive in character. All changes and modifications that come within the spirit of the invention are desired to be protected and are to be considered within the scope of the disclosure.

1. A spinal implant for insertion into an intervertebral disc space, the implant comprising:
a leading end;
a trailing end; and
a flexible mid section connecting the leading end and the trailing end;
wherein the implant is deformable at the flexible mid section to thereby permit a substantially straight entry of the implant into a selected disc space.

2. The implant of claim 1, wherein the implant is delivered to the selected disc space at a desired insertion angle of approach.

3. The implant of claim 2, wherein the leading end comprises a bullet shaped configuration.

4. The implant of claim 3, further comprising a central implant aperture bounded by the leading end, the trailing end and the flexible mid section.

5. The implant of claim 2, wherein the desired insertion angle of approach is between 5 to 45 degrees.

6. The implant of claim 2, wherein the implant is delivered to the disc space via a lateral approach.

7. The implant of claim 1, wherein the selected disc space is L4-L5 or L5-S1.

8. The implant of claim 1, wherein the flexible mid section becomes substantially rigid after the implant is inserted in the disc space.

9. The implant of claim 1, wherein the flexible mid section is comprised of one or more of a flexible metallic section, at least one pivoting connection and at least one implant slot.

10. An intervertebral spinal implant, the implant comprising:
a curved leading end;
a trailing end; and
a flexible mid section connecting the curved leading end and the trailing end;
wherein the spinal implant is delivered at a desired insertion angle of approach and is deformable about the flexible mid section to thereby permit substantially straight entry of the implant into a selected disc space.

11. The implant of claim 10, wherein the curved leading end comprises a bullet shaped configuration.

12. The implant of claim 11, further comprising a central implant aperture bounded by the curved leading end, the trailing end and the flexible mid section.

13. The implant of claim 10, wherein the desired insertion angle of approach is between 10 to 30 degrees.

14. The implant of claim 10, wherein the implant is delivered to the disc space via a lateral approach.

15. The implant of claim 10, wherein the selected disc space is L4-L5 or L5-S1.

16. The implant of claim 10, wherein the flexible mid section becomes substantially rigid after the implant is inserted in the disc space.

17. The implant of claim 10, wherein the flexible mid section is comprised of one or more of a flexible metallic section, at least one pivoting connection and at least one implant slot.

18. A spinal implant for insertion into a selected disc space, the implant comprising:
leading end;
a trailing end; and
a flexible mid section connecting the leading end and the trailing end;
wherein the implant is delivered at a desired insertion angle of approach via an implant insertion channel;
wherein the implant is deformable about the flexible mid section through interaction with the implant insertion channel to thereby permit substantially straight lateral entry of the implant into the selected disc space via a lateral approach;
wherein the flexible mid section permits the implant reach an equilibrium position in the disc space after implant insertion through motion of the flexible mid section;
wherein the flexible mid section becomes substantially rigid after the implant insertion inserted in the disc space.

19. The implant of claim 18, wherein the flexible mid section is comprised of one or more of a flexible metallic section, at least one pivoting connection and at least one implant slot.

20. The implant of claim 18, wherein the implant insertion channel comprises a first end, a turn section and a second end.
